(19) United States
(12) Patent Application Publication
(10) Pub. No.: US 2005/0260265 A1
(43) Pub. Date: Nov. 24, 2005

(54) Mesostructured silica/block copolymer monoliths as a controlled release device and methods of manufacture

(76) Inventor: Michael D. Wyrsta, Santa Barbara, CA (US)

Correspondence Address: Michael A. O'Neil;Michael A. O'Neil, P.C., Suite 820, 5949 Sherry Lane, Dallas, TX, 75225, US

(21) Appl. No.: 11/134,572

(22) Filed: May 19, 2005

Related U.S. Application Data
(60) Provisional application No. 60/573,054, filed on May 20, 2004.

(51) Int. Cl. A61K 9/22; A61K 9/16; A61K 9/50; A61K 33/24

(52) U.S. Cl. 424/468; 424/617

(57) ABSTRACT

The invention comprises the design, synthesis, and characterization of mesostructured silica/block copolymer composite monoliths as controlled release systems. The controlled release function is based on the formation of mesostructured silica/block copolymer architectures via surfactant-templated sol-gel processing. Multi-layered or gradient monoliths are produced by layer-by-layer sol-gel processing to provide pulsed and programmed release characteristics. A simple, rapid route to prepare combinatorial compositional monolith libraries provides high-throughput synthesis and rapid screening of the release characteristics of the monoliths.

### CLAIM OF PRIORITY

Applicant claims priority based on provisional patent application Ser. No. 60/573,054, filed May 20, 2004.

### TECHNICAL FIELD

The present invention relates to a mesoporous polymer/inorganic oxide hybrid material host composition for controlled release of molecular species. More particularly the invention relates to a film, fiber, monolith, powder or coating composed of a mesoporous polymer/inorganic oxide hybrid material host for use as a controlled release media.

### BACKGROUND OF THE INVENTION

When a chemical substance is incorporated into a solid material, a controlled release system is important in order to facilitate release of the chemical substance at a designated rate. Controlled release systems are particularly needed in the medical field where controlled drug delivery is required and various other industrial applications where a controlled chemical release is required such as agricultural chemical applications, cosmetics, and catylsis.

Heretofore, various techniques and materials have been proposed for a controlled release system, but most are directed to mixing a chemical substance into a polymer gel or forming a complex consisting of a chemical substance and a polymeric material such as organic poly(lactic acid) or wholly inorganic material such as porous silica. Although silica gels are versatile and can incorporate vrious types of chemical substances therein, the release from the silica matrix practices a diffusion release mechanism and therefore rapidly decreases.

In recent years a liquid solution or a coating film, which comprises surfactant molecules as the main component, have begun to emerge as promising controlled release materials. As a result of trends toward more complex controlled release materials with the proper release profile and safety, polymer surfactant molecules have been rigorously researched and have found use as such controlled release agents.

However, many of the polymer coatings and formulations used in controlled release applications lack the ability to tune the release profile of the encapsulated molecular species. Further, the polymer erodes and the chemical substance is released into the environment. Also many controlled release formulations are liquid and therefore lose their ability to control the release of their contents upon dilution.

What is needed is a release device with release characteristics which can be easily tuned over a wide range.

### SUMMARY OF THE INVENTION

The present invention comprises a design, synthesis, and characterization of mesostructured silica/block copolymer composite in the form of a film, powder, monolith, or fiber as controlled release systems capable of giving a material having low toxicity and tunable profile of release of contents which overcomes the foregoing and other difficulties which have long since characterized the prior art. In accordance with the broader aspects of the invention, the present invention relates to the formation of mesostructured silica/block copolymer monolith architecture for obtaining a controlled release rate using the silica matrix and a polymer which can be eroded or eluted from the matrix. By controlling the formed silica/polymer architecture, the release characteristics can be modified in a wide range. In accordance with more specific aspects of the invention, surfactant-directed silicate polymerization is a suitable method to form silica/polymer architectures. The obtained silica/polymer composites are called mesostructured silica, which were first reported in 1992 and have attracted a great deal of interest in synthesis study and applications exploration. The sol-gel based polymer self-assembly and silicate polymerization offer control over the silica/polymer architectures, which is expected to significantly enhance the control of the doped compound therein, for example, a dye. Therefore, mesostructured silica has been recognized as potential advanced optical materials, particularly as host media for molecules and complexes exhibiting optical functionalities. The use of nonionic-surfactant as structure-directing agent (SDA) and acidic condition for polymerization allow a wide range of compositions, mesoscopic structures and morphologies to tailor mesostructured silica with desired properties. Moreover, the nonionic surfactants used in mesostructured materials synthesis, generally Pluronic block copolymers, have been be used for drug delivery because of the fact that the core-shell architecture of Pluronic micelles are efficient carriers for compounds. The additional silica matrices in the present invention contribute greatly to the enhanced storage property by maintaining the micelles in a dispersed state, as well as by increasing the incorporation ability of various therapeutic reagents, which alone exhibit poor solubility, undesired pharmacokinetics and low stability in a physiological environment.

The controlled release from silica monoliths is based on modifying the polymer elution rate and the matrix diffusion rate. The rate of and duration of compound release can be controlled over a wide range by many factors, including matrix composition, physical structure of the system, morphology, the release media and the physicochemical properties of the compound itself. The advantages of proposed release device also include supporting very long release duration, easily removable, various morphologies (monolith, film) for further fabrication and versatile for the incorporation of molecules with different physicochemical properties. Moreover, in combination with a layer-by-layer sol-gel processing approach, multi-layered or gradient monoliths can be produced, indicating potential applications in pulsed and programmed release. The present invention comprises a general method to fabricate a controlled-release device which is compatible with various active agents to offer modified release dynamics. Further, the present invention comprises a simple, rapid route to produce combinatorial compositional monolith libraries for the high-throughput synthesis and screening of monoliths with the desired release characteristics, which can be extended to the preparation of multi-layered or gradient monoliths.

The resulting mesoporous polymer/inorganic oxide hybrid material host can be applied to applications requiring a controlled release of a molecular entity(s) such as oral delivery of human and non-human therapeutics, coated biomedical devices, the dispersal delivery agent for agriculturally relevant molecules, and various personal care and food products.

### BRIEF DESCRIPTION OF THE DRAWINGS

A more complete understanding of the present invention may be had by reference to the following Detailed Description when taken in connection with the accompanying Drawings, wherein:

FIG. 1 illustrates one step in the preparation of the mesostructured silica/block copolymer monolith of the present invention;

FIG. 2 illustrates the controlled release of the doped mesostructured silica/block copolymer monolith of the present invention;

FIG. 3A is a graphical representation of the differential release profiles of Rhodamine 6G having mesostructured silica/block copolymer monolith of the present invention contained therein;

FIG. 3B is a graphical representation similar to FIG. 3A showing the cumulative release profiles of Rhodamine 6G having mesostructured silica/block copolymer monolith of the present invention contained therein;

FIG. 4 is a graphical representation of peaks of the differential release patterns as they relate to the polymers themselves and water-soluble molecules remaining in the monolith;

FIG. 5A is a graphical representation of the present release profile of a selected block copolymer;

FIG. 5B is a graphical representation similar to FIG. 5A showing the present release profile of another block copolymer; and

FIG. 5C is a graphical representation similar to FIGS. 5A and 5B showing the present release profile of yet another block copolymer.

### DETAILED DESCRIPTION

### EXPERIMENTAL DEVELOPMENT OF THE PRESENT INVENTION

The chemical reagents used for the synthesis include the following: Pluronic L64 (EO13PO30EO13, Mav=2900, PEO wt %=40%; Aldrich), Pluronic P84 (EO19PO43EO19, Mav=4200, PEO wt %=40%; Aldrich), Pluronic P104 (EO27PO61EO27, Mav=5900, PEO wt %=40%; Aldrich), Pluronic F88 (EO104PO39EO104, Mav=11400, PEO wt %=80%, Aldrich), and tetraethyl-orthosilicate (TEOS, Merck). The fluorescence dyes employed in this study were Rhodamine 6G (Molecular Probe) and LD 490 (Exciton).

Sample Preparation

1. Preparation of Dye-Containing Mesostructured Silica/Block Copolymer Composite Monolith

Dye-containing mesostructured silica/block copolymer composite monoliths were prepared in standard 96-well plate through an evaporation-induce self-assembly (EISA) sol-gel processing, as follows. A block copolymer was dissolved in a sol of TEOS/water/ethanol that was pre-hydrolyzed at 60° C. for 2 hours, forming a homogeneous solution, followed by transferring to a standard 96-well plate for gelation of monoliths. In each well, 200 μl sol and 5 μl dye (1 μ mol) were pipetted. These monoliths were gelled and dried at ambient environment for 3 days and at 60° C. oven for 1 day.

2. Library Design

Combinatorial compositional monolith libraries were prepared for high-throughput synthesis and screening of the monolith with desired release characteristics. L64, P84, P104 and F88 were used in this work. For the library of each block polymer, the initial polymer mass content was 0%, 1%, 3%, 5%, 7%, 10%, 15% and 20%, and the molar composition was TEOS:HCl(pH=2):ethanol=1:(4, 8, 12):(4, 12, 20).

3. Release Set-Up

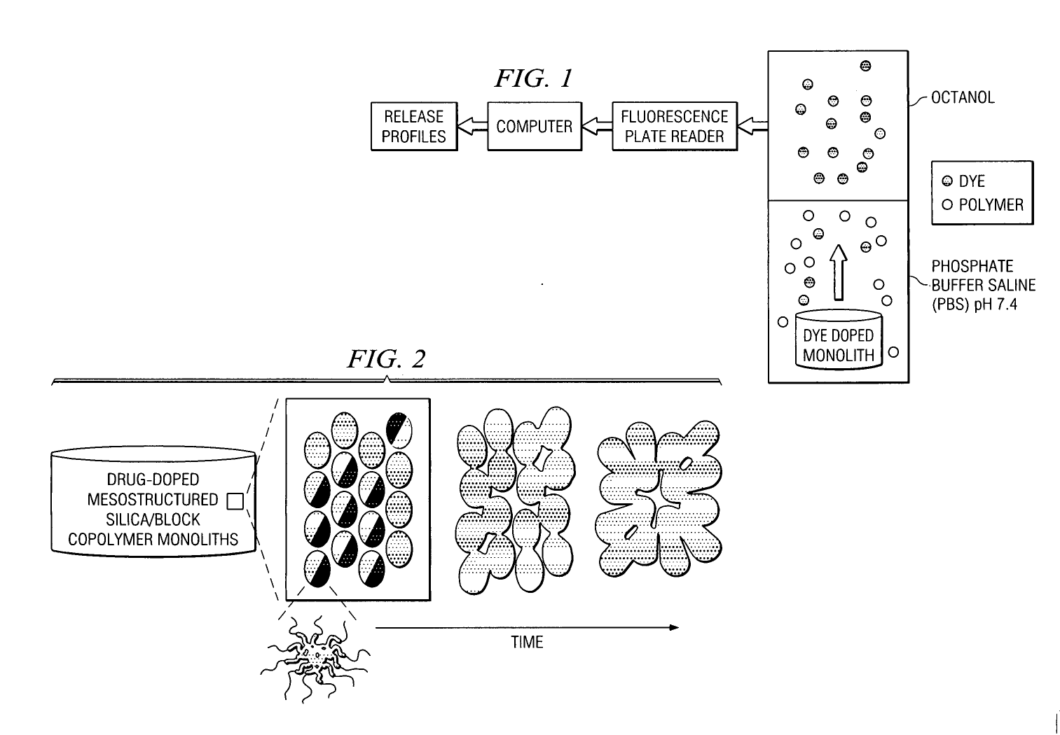

The set-up of the release profiles is illustrated in FIG. 1. The fluorescent dye doped in the monoliths was released in the wells of a 96-well deep plate. The release medium was aqueous buffer with a pH of 7.4 at a temperature of 25° C. and octanol was introduced to extract released dye for concentration analysis. For each well, a piece of monolith was immersed in 400 μl of aqueous buffer (pH=7.4), followed by adding 600 μl of octanol.

### ANALYTICAL METHOD OF THE PRESENT INVENTION

1. Investigation of Release Profiles

In this study, fluorescent dye, Rhodamine 6G and LD 490, were employed as model compound for release. The amount of released dye was determined by monitoring changes of fluorescence intensity, which was measured using a fluorescence plate reader (HTSoft 7000; PerkinElmer) (485 nm excitation, 595 nm emission for Rhodamine 6G and 430 nm excitation, 535 nm emission for LD 490). For a typical procedure, 5 μl solution of the octanol layer was transferred to the well of a standard 96-well plate, followed by adding 195 μl 5:1 volume ratio of ethanol/water for dilution. A series of standard solutions that were comprised of known concentrations of fluorescence dye were pipetted to the remaining wells of the plate as reference. The samples were rotated for 1 minute at 25° C. Precise readings of the well's fluorescence and then reference curves based on the fluorescence response of standard solutions were to quantitatively calculate the released amount of dye from these monoliths.

2. Determination of Model Compound Content

To determine model compound content and remaining amount dyes after release, the dye-doped monolith was dissolved in 10 ml 2M NaOH with 1:1 ethanol/water (v/v) by overnight rotation; then a 400 μl volume of the above solution was pipetted to a well of a deep plate and followed by 600 μl octanol to extract the dye. The dye content then can be determined by the method described above.

### CHARACTERIZATION METHODS OF THE PRESENT INVENTION

Release profiles were investigated by a Perkin Elmer HT Soft 7000 Plus Bio Assay Reader, which is designed for luminescence and adsorption readings of various microplates. For fluorescence analysis, the excitation wavelength used was 485 nm and analysis wavelength was 595 nm for Rhodamine 6G, and for LD 490, they were 430 nm and 535 nm. X-ray diffraction (XRD) patterns were obtained on a Scintag PAD X diffractometer employing Cu Ka radiation. Transmission electron microscopy (TEM) was performed a JEOL 2000 FX after drying of samples at 373 K for 4 hours.

Results and Discussion

The concept of controlled release of the doped mesostructured silica/block copolymer monolith is shown in FIG. 2. The incorporated dyes are located in the polymer phase of the silica polymer architecture, which is powerful in incorporating and stabilizing not only hydrophilic but also hydrophobic molecules and DNAs. In the release process, water molecules penetrate the silica framework and erode the polymer to release the dyes. Dye molecules are then diffused with the eroded polymer from the silica framework. The release rate is determined by the polymer eroding rate, which can be controlled through some factors.

A dye containing mesostructured silica/block copolymer monoliths demonstrated an evident color difference of the dye content. A gradient monolith prepared by layer-by-layer method demonstrated an evident color change, demonstrating the concentration of dye is gradually changed along the axis or radius because of the diffusion between the interfaces.

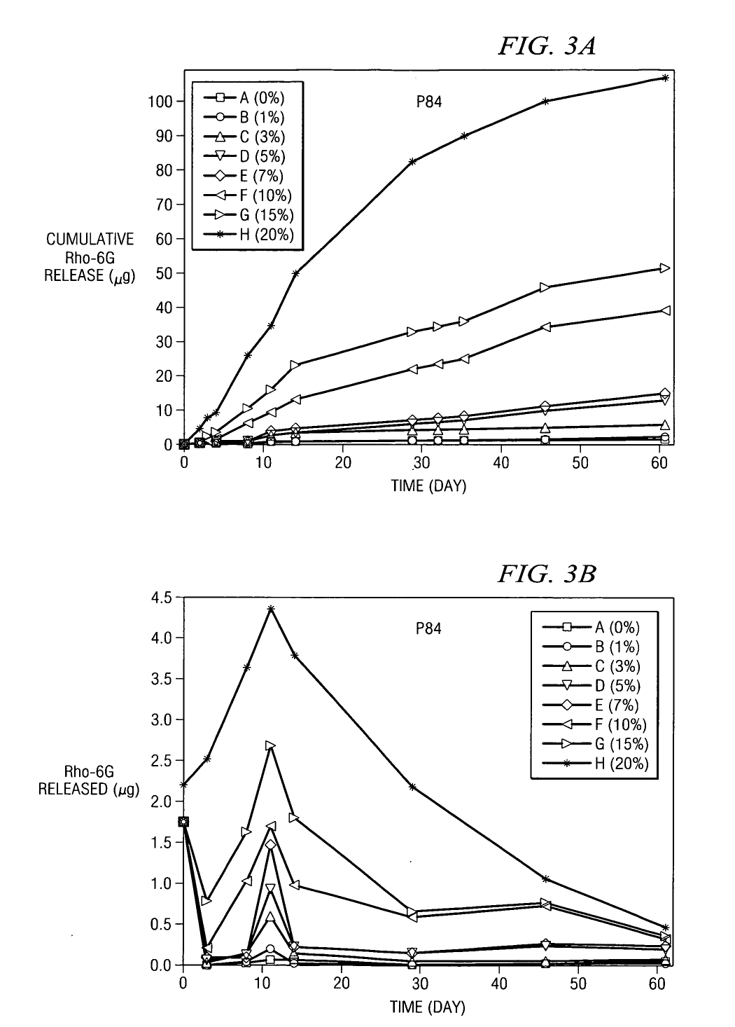

FIGS. 3A and 3B show the typical cumulative and differential release profiles of Rhodamine 6G-containing mesostructured silica/block copolymer monoliths with different polymer concentrations (0%-20%) for the first 2 months. These monoliths were doped with same amount of Rhodamine 6G (480 μg per monolith, which was confirmed by the dye-content-determination experiment) and prepared by Pluronic P84 and a sol of TEOS:HCl (pH=2):ethanol=1:4:4 (molar ratio) through sol-gel processing. The initial P84 mass concentrations of the monoliths are 0%, 1%, 3%, 5%, 7%, 10%, 15% and 20%. The cumulative release profiles shown in FIG. 3A demonstrate the feasibility of modifying the release rate by tuning the polymer concentrations in the monolith. The release rate increased with respect to the increasing polymer concentrations. After 2 months, released Rhodamine 6G varied from 1.7 μg to 107 μg.

Referring to FIG. 3B, more release characteristics are shown in the differential release pattern, facilitating clarification of the release dynamics. Three-phase mode release profiles were evident with an initial burst reaching 0.07 μg in the first hour due to the surface localized dye molecules, followed by a decline through day 3. The release rate increased again at around day 11 and then decreased slowly or reached a plateau through day 62. The release pattern of Rhodamine 6G in mesostructured monolith does not have a high burst release nor the occurrence of an extended period of little or no release. This release pattern indicated a simultaneous occurrence of matrix diffusion and polymer elution, as compared with the purely diffusion-controlled release kinetics calculated from the classical Higuchi equation. The release rate will clearly increase with time by the increasing dye permeability of the system with progressive polymer elution. This is the second phase of the release pattern.

Similar results occure during polymer bulking eroding process, in which water uptake by the system is much faster than polymer eroding. However, after a certain time period, this effect is overcompensated by a diffusion-controlled release, due to increasing diffusion pathlengths of polymers and dyes. Thus, the release rate will slowly decrease or reaches a plateau, which is recognized as the third phase of the release pattern.

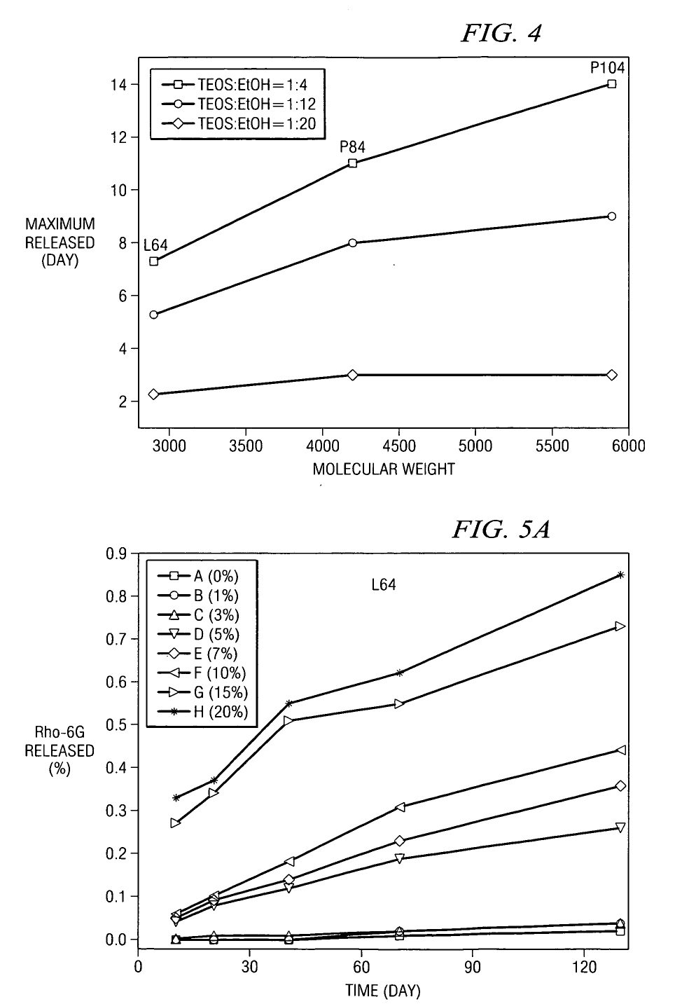

The peak in the differential release pattern reflects the transition from phase 2 to phase 3, which is influenced by the factors of monolith composition. The position of peaks relates to the polymers themselves and water-soluble molecules remaining in the monolith as shown in FIG. 4. For polymers with same PPO content, the smaller molecular weight, the easier the polymer is eluted. Further, for a monolith with much ethanol remaining, the monolith wil 1 hydrate more rapidly than it will be eluted. For example, in FIG. 4, with increasing molecular weight of L64, P84 and P104, the peaks appeared on day 7.3, 11 and 14, respectively. Increasing the mole ratio of ethanol/TEOS, the peak appeared at an earlier day. In the condition of TEOS/ethanol=1:20, the peaks of L64, P84 and P104 appeared at the nearly same day, which can accounted for too much ethanol resulting in very fast hydration and elution. The height of the peak is therefore influenced by the polymer concentration, which corresponds to the dye permeability during polymer elution.

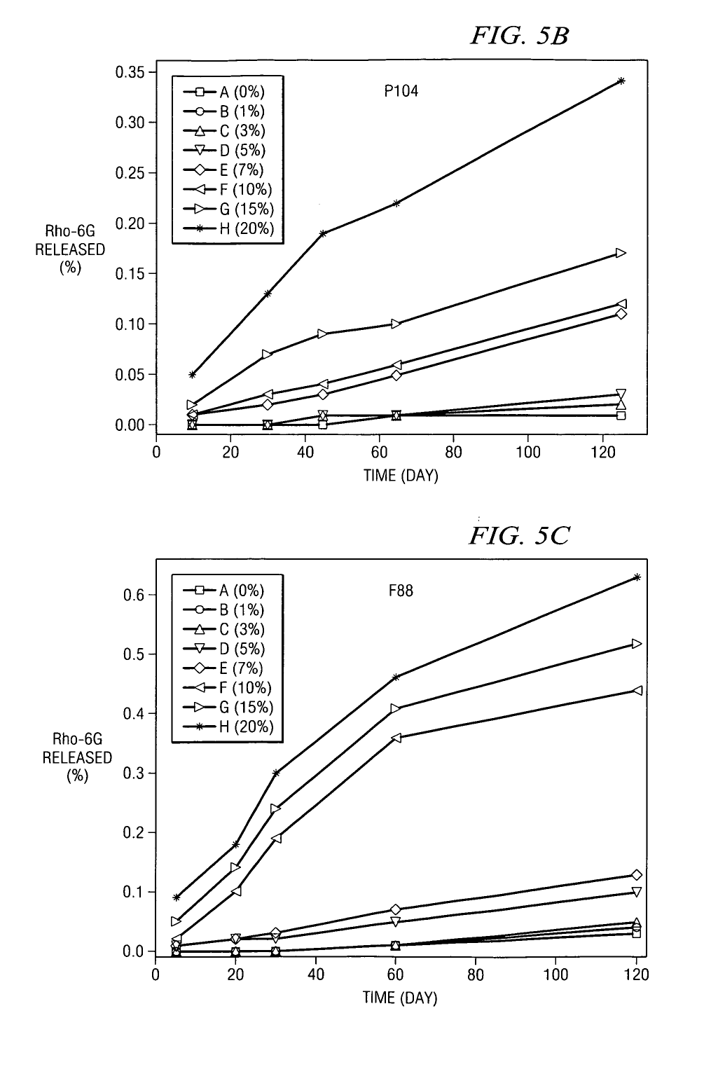

The percent release profiles of different block copolymers are shown in FIGS. 5A, 5B, and 5C. The overall release of L64 after a release duration of 130 days varied from 2% to 85%, and for P84, varied from 1% to 34%; for F88, varied from 3% to 63%. These results can be explained by the polymer elution process. L64 is smaller than P84 and F88 has larger hydrophilic section percentage (80%) over P84 (40%). Therefore, L64 and F88 elute faster than P84 and finally, will have better release characteristics.

The release profiles can be modified over a wide range, which means a release map can be established based on the combinatorial composition monolith libraries. A monolith with desired release characteristics can be easily located and then prepared according to this release map. The modified release characteristics include tuning the release rate, duration and dynamics. These objectives can be obtained through the controlling the following factors:
1) the effect of block copolymer concentration on dye release as shown in FIGS. 3, 5A, 5B, and 5C; and
2) the effect of block copolymers on dye release as shown in FIGS. 4, 5A, 5B, and 5C.
   Ultimately, the release duration can be tuned with different polymers.

The controlled release function of the monolith is based on the forming of ordered silica/polymer architectures. It has previously been demonstrated that the evaporation-induced self-assembly (EISA) technique results in optically clear monoliths with an ordered mesophase. The mesostructured ordering of the dye-containing monoliths were characterized by low-angel X-ray diffraction (XRD) and transmission electron microscopy (TEM). As shown in FIG. 4, XRD peaks were observed at low angles, which are more and sharper with respect to the increasing polymer concentration. Using combination of the XRD data and TEM, structures of monoliths with different polymer concentration are clearly understood. For monoliths with 0%, 1% and 3% P84, that is, trace a, b and c (not shown in the XRD figure), with polymer concentrations below cmc, no peaks were observed in the XRD figure, which means there was limited formation of silica/polymer monodispersed structure in these monoliths. For monoliths with 5%, 7% and 10% P84, that is, trace d, e, and f, a broad peak was shown in the XRD pattern. The TEM images shown in FIGS. 5A, 5B, and 5C show typical worm-like structures. In this case the silica/polymer architectures were formed but limited long range order. In the XRD pattern of monoliths with 15% and 20% P84 (trace g and h), three peaks were observed that could be indexed as the (100), (110) and (200) reflections of a hexagonal mesostructure (p6mm), which is also confirmed by TEM. With the increasing polymer concentration, the XRD peaks shifted to a lower angle, indicating a lager unit cell parameter. The XRD pattern and TEM image shown in FIGS. 5A, B, and C belong to the monolith after 2 months release. The preservation of the mesostructured ordering demonstrates that the polymer/silica matrix of the composite monolith remains stable after release of incorporated dyes.

Although preferred embodiments of the invention have been illustrated in the accompanying Drawings and described in the foregoing Detailed Description, it will be understood that the invention is not limited to the embodiments disclosed, but is capable of numerous rearrangements, modifications, and substitutions of parts and elements without departing from the spirit of the invention.

1. A composition for controlled release of molecular species comprising:
(A) a product of the hydrolysis and condensation of an organic polymer and at least one metal alkoxide compound performed in the presence of a catalyst;
(B) the metal alkoxide selected from the group consisting of:
(1) a compound represented by the formula RₐM(OR¹)ₓ₋ₐ wherein
(a) R represents a variable selected from a group consisting of a hydrogen atom, a halogen atom, or an organic group;
(b) R¹ represents an organic group;
(c) a is an integer of the group consisting of 1 and 2; and
(d) x represents metal or metalloid dependent electronic valency;
(2) a compound represented by the formula M(OR²) wherein R² represents an organic group; and
(3) a compound represented by the formula R³_{b}(R⁴O)_{x-b}M-(R⁷)_{d}-M(OR⁵)_{x-c}R⁶_{c} wherein
(a) R³, R⁴, R⁵, and R⁶ represent an organic group;
(b) b and c are an integer selected from the group consisting of 1 and 2;
(c) R⁷ represents a variable selected from a group consisting of an oxygen atom, an organic group, a combination of an oxygen atom and an organic group, and a group represented by —(CH₂)ₙ— wherein n is selected from a group of integers from 1 to 1,000,000; and
(d) d is selected from the group consisting of 0 and 1;
(C) a product of hydrolysis and condensation obtained by hydrolizing and condensing an organic polymer and at least one metal compound performed in the presence of a catalyst; and
(D) the metal compound selected from the group consisting of:
(1) a compound represented by the formula RₐM(OR¹)ₓ₋ₐ wherein
(a) R represents a variable selected from the group consisting of a hydrogen atom, a halogen atom, or an organic group;
(b) R¹ represents an organic group;
(c) a is an integer of the group consisting of 1 and 2; and
(d) x represents metal or metalloid dependent electronic valency;
(2) a compound represented by the formula M(OR²) wherein R² represents an organic group; and
(3) a compound represented by the formula R³_{b}(R⁴O)_{x-b}M-(R⁷)_{d}-M(OR⁵)_{x-c}R⁶_{c} wherein
(a) R³, R⁴, R⁵, and R⁶ represent an organic group;
(b) b and c are an integer selected from the group consisting of 1 and 2;
(c) R⁷ represents a variable selected from the group consisting of an oxygen atom, an organic group, a combination of an oxygen atom and an organic group, and a group represented by —(CH₂)ₙ— wherein n is selected from a group of integers from 1 to 1,000,000; and
(d) d is selected from the group consisting of 0 and 1.

2. The composition according to claim 1 wherein the hydrolysis and condensation of an organic polymer and at least one metal alkoxide compound is performed in the presence of an acid catalyst.

3. The composition according to claim 1 wherein the hydrolysis and condensation of an organic polymer and at least one metal alkoxide compound is performed in the presence of an base catalyst.

4. A composition for controlled release of molecular species comprising:
(A) a product of the hydrolysis and condensation of an organic polymer and at least one metal alkoxide compound performed in the presence of a catalyst;
(B) the metal alkoxide selected from the group consisting of:
(1) a compound represented by the formula RₐM(OR¹)ₓ₋ₐ wherein
(a) R represents a variable selected from the group consisting of a hydrogen atom, a halogen atom, or an organic group;
(b) R¹ represents an organic group;
(c) a is an integer of the group consisting of 1 and 2; and
(d) x represents metal or metalloid dependent electronic valency;
(2) a compound represented by the formula M(OR²) wherein R² represents an organic group; and
(3) a compound represented by the formula R³_{b}(R⁴O)_{x-b}M-(R⁷)_{d}-M(OR⁵)_{x-c}R⁶_{c} wherein
(a) R³, R⁴, R⁵, and R⁶ represent an organic group;
(b) b and c are an integer selected from the group consisting of 1 and 2;
(c) R⁷ represents a variable selected from the group consisting of an oxygen atom, an organic group, a combination of an oxygen atom and an organic group, and a group represented by —(CH₂)ₙ— wherein n is selected from a group of integers from 1 to 1,000,000; and
(d) d is selected from the group consisting of 0 and 1;
(C) a product of the hydrolysis and condensation of an organic polymer and at least one metalloid compound performed in the presence of a catalyst; and
(D) the metalloid compound selected from the group consisting of:
(1) a compound represented by the formula RₐM(OR¹)ₓ₋ₐ wherein
(a) R represents a variable selected from the group consisting of a hydrogen atom, a halogen atom, or an organic group;
(b) R¹ represents an organic group;
(c) a is an integer of the group consisting of 1 and 2; and
(d) x represents metal or metalloid dependent electronic valency;
(2) a compound represented by the formula M(OR²) wherein R² represents an organic group; and
(3) a compound represented by the formula R³_{b}(R⁴O)_{x-b}M-(R⁷)_{d}-M(OR⁵)_{x-c}R⁶_{c} wherein
(a) R³, R⁴, R⁵, and R⁶ represent an organic group;
(b) b and c are an integer selected from the group consisting of 1 and 2;
(c) R⁷ represents a variable selected from the group consisting of an oxygen atom, an organic group, a combination of an oxygen atom and an organic group, and a group represented by —(CH₂)ₙ— wherein n is selected from a group of integers from 1 to 1,000,000; and
(d) d is selected from the group consisting of 0 and 1.

5. The composition according to claim 4 wherein the hydrolysis and condensation of an organic polymer and at least one metal alkoxide compound is performed in the presence of an acid catalyst.

6. The composition according to claim 4 wherein the hydrolysis and condensation of an organic polymer and at least one metal alkoxide compound is performed in the presence of an base catalyst.
